(19) United States
(12) Patent Application Publication
(10) Pub. No.: US 2004/0185101 A1
(43) Pub. Date: Sep. 23, 2004

(54) Biodegradable triblock copolymers as solubilizing agents for drugs and method of use thereof

(75) Inventors: Chung Shih, Sandy, UT (US); Gaylen Zentner, Salt Lake City, UT (US); Ai-zhi Piao, Salt Lake City, UT (US); Kirk Dee Fowers, Layton, UT (US)

Correspondence Address: THORPE NORTH & WESTERN, LLP., 8180 SOUTH 700 EAST, SUITE 200, P.O. BOX 1219, SANDY, UT, 84070, US

(73) Assignee: MacroMed, Incorporated.

(21) Appl. No.: 10/734,740

(22) Filed: Dec. 11, 2003

Related U.S. Application Data
(63) Continuation-in-part of application No. 09/971,074, filed on Oct. 3, 2001, now abandoned.
(60) Provisional application No. 60/279,363, filed on Mar. 27, 2001.

(51) Int. Cl. A61K 9/14

(52) U.S. Cl. 424/486

(57) ABSTRACT

Biodegradable ABA-type or BAB-type triblock copolymers are disclosed that, at functional concentrations, are capable of solubilizing drugs, especially hydrophobic drugs, in a hydrophilic environment to form a solution at temperatures relevant for parenteral and particularly for intravenous administration as well as all other routes of administration benefiting from an aqueous drug solution. The copolymers are comprised of about 50.1 to 65% by weight of biodegradable hydrophobic A polymer block(s) comprising a biodegradable polyester, and about 35 to 49.9% by weight of a biodegradable hydrophilic B polymer block comprising a polyethylene glycol (PEG), and wherein the triblock copolymer has a weight-averaged molecular weight of between about 1500 to 3099 Daltons.

This application is a continuation-in-part of Ser. No. 09/971,074, filed Oct. 3, 2001, which in turn claims benefit of Ser. No. 60/279,363, filed Mar. 27, 2001.

### FIELD OF THE INVENTION

The present invention relates to biodegradable triblock copolymers having a high weight percentage (at least 50 percent) of hydrophobic block(s) and low molecular weight (1500-3099 Daltons), and their use for solubilizing a hydrophobic drug in a hydrophilic environment. The triblock copolymers of the present invention exist as high viscosity liquids in neat form and form solutions in aqueous environments at body temperature and are suitable for parenteral and particularly for intravenous (I.V.) delivery. Therefore, the triblock copolymers of the present invention can be used as solubilizing agents for drugs that are substantially insoluble in water, or as solubilizing agents for drugs that require enhancement of their water solubility.

### BACKGROUND OF THE INVENTION

Many important drugs have limited solubility in water, especially hydrophobic drugs. In order to attain the full expected therapeutic effect of such drugs, it is usually required that a solubilized form of the drug be administered to a patient. Recently, many peptide/protein drugs, effective for a variety of therapeutic applications, have become commercially available through advances in recombinant DNA and other technologies. Many peptide drugs are of limited solubility and/or stability in conventional liquid carriers and are therefore difficult to formulate and administer.

A number of methods for solubilizing drugs have been developed and most of them are based on the use of solvents or cosolvents, surfactants, complexing agents (for example, cyclodextrins or nicotinamide), or use of complicated drug carriers (for example, liposomes). Each of the above methods has one or more particular drawbacks. For example, the use of conventional surfactants and cyclodextrins to solubilize hydrophobic drugs has drawbacks related to surfactant and cyclodextrin toxicity and/or precipitation of the solubilized drugs once administered to the patient or when otherwise diluted in an aqueous environment.

Amphiphilic block copolymers are potentially effective drug carriers that are capable of solubilizing drugs, especially hydrophobic drugs, into an aqueous environment. For example, there have been reported many studies on amphiphilic block copolymers having surfactant-like properties, and particularly noteworthy are the attempts to incorporate hydrophobic drugs into block copolymers which are stabilized due to the specific nature and properties of the copolymer. For example, EP No. 0 397 307 A2 (See also EP No. 0 583 955 A2 and EP No. 0 552 802 A2.) disclose polymeric micelles of an AB type amphiphilic diblock copolymer which contains poly(ethylene oxide) as the hydrophilic component and poly(amino acids) as the hydrophobic component, wherein therapeutically active agents are covalently bonded to the hydrophobic component of the polymer. Although this polymeric micelle is provided as a means of administering a hydrophobic drug, it is disadvantageous in that it requires the introduction of functional groups into the block copolymer, and the covalent coupling of the drug to the polymeric carrier.

U.S. Pat. No. 4,745,160 discloses a water insoluble, pharmaceutically or veterinary acceptable amphiphilic, non-crosslinked linear, branched or graft block copolymer having polyethylene glycol as the hydrophilic component and poly(D-, L-, and D,L-lactic acids) as the hydrophobic components. Although the block copolymer is an effective dispersing or suspending agent for a hydrophobic drug, the block copolymer is insoluble in water and has a molecular weight of 5,000 or more. Furthermore, the hydrophilic component is at least 50% by weight based on the weight of the block copolymer and the molecular weight of the hydrophobic component is 5,000 or less. In the preparation process, a water-miscible and lyophilizable organic solvent is used. When a mixture of the polymer, the drug, and an organic solvent are mixed with water, precipitates are formed and then the mixture is directly lyophilized to form particles. Therefore, when this particle is dispersed in water, it forms a colloidal suspension containing fine particles wherein hydrophilic components and hydrophobic components are mixed.

U.S. Pat. No. 5,543,158 discloses nanoparticles or microparticles formed from a block copolymer consisting essentially of poly(alkylene glycol) and a biodegradable polymer, poly(lactic acid). In the nanoparticle or microparticle, the biodegradable moieties of the copolymer are in the core of the nanoparticle or microparticle and the poly(alkylene glycol) moieties are on the surface of the nanoparticle or microparticle in an amount effective enough to decrease uptake of the nanoparticle or microparticle by the reticuloendothelial system. In this patent, the molecular weight of the block copolymer is high and the copolymer is insoluble in water. A nanoparticle is prepared by dissolving the block copolymer and a drug in an organic solvent, forming an o/w emulsion by sonication or stirring, and then collecting the precipitated nanoparticles containing the drug. It does not provide for the solubilization of hydrophobic drugs. The nanoparticles prepared in this patent are solid particles that are dispersed in water.

Currently there are few synthetic or natural polymeric materials which can be used for the controlled delivery of drugs, including peptide and protein drugs, because of strict regulatory compliance requirements, such as biocompatibility and low toxicity, having a clearly defined degradation pathway, and safety of the degradation products. The most widely investigated and advanced biodegradable polymers in regard to available toxicological and clinical data are the aliphatic poly(α-hydroxy acids), such as poly(D-, L-, or D,L-lactic acid) (PLA) and poly(glycolic acid) (PGA) and their copolymers (PLGA). These polymers are commercially available and are presently used as bioresorbable sutures. An FDA-approved system for controlled release of leuprolide acetate, Lupron Depot™, is also based on PLGA copolymers. Lupron Depot™ consists of injectable microspheres, which release leuprolide acetate over a prolonged period (e.g., about 30 to 120 days) for the treatment of prostate cancer. Based on this history of use, PLGA copolymers have been the materials of choice in the initial design of parenteral controlled release drug delivery systems using a biodegradable carrier.

Even though there has been some limited success, these PLA, PGA, and PLGA polymers present problems as drug carriers that are associated with their physicochemical properties and attendant methods of fabrication. Hydrophilic macromolecules, such as polypeptides, cannot readily diffuse through the hydrophobic matrices or membranes of polylactides. Drug loading and device fabrication using PLA and PLGA often requires use of toxic organic solvents or high temperatures. Also, the geometry of the administered solid dosage form may mechanically induce tissue irritation and damage.

U.S. Pat. Nos. 6,004,573; 6,117,949 and 6,201,072 disclose low molecular weight, biodegradable triblock copolymers having a high weight percentage (e.g., at least 50 weight percent) of hydrophobic block(s) as solubilizing agents for drugs and hydrophobic drugs in particular. These patents disclose polymeric delivery systems, having reverse thermal gelation properties, and are free of many of the problems mentioned above. These patents show that certain amphiphilic, biodegradable triblock copolymers that form thermal gels and have a high weight percentage (at least 50 weight percent) of hydrophobic block(s) and are covalently attached to poly(ethylene oxide) are very effective in solubilizing drugs and in particular hydrophobic drugs. The resulting composition of triblock copolymers and water results in the drug being dissolved by the action of the triblock copolymers thereby enhancing the efficiency and facilitating administration of a uniform and accurate dose which may then, in many cases, enhance the therapeutic effects of the drug. Controlling the molecular weights, composition, and relative ratios of the hydrophilic and hydrophobic blocks may optimize such solubilizing effects. However, the block copolymers disclosed in these patents possess reverse thermal gelation properties wherein the sol/gel transition temperature is generally lower than a temperature required for I.V. delivery purposes of between at least 35-42° C.

### SUMMARY OF THE INVENTION

The present invention provides a biodegradable polymeric composition capable of solubilizing a drug, and most notably, a hydrophobic drug into a hydrophilic environment. This composition may then be used in preparing a free flowing solution of such drugs suitable for intravenous delivery and also the delivery of drugs by any other route where administration of a drug solution is desired.

The present invention also provides a method for effectively solubilizing a drug, including a hydrophobic drug being solubilized into a hydrophilic environment, and a method for effectively administering such a drug to animals by intravenous (I.V.) delivery. However, any other means, such as parenteral, ocular, topical, inhalation, transdermal, vaginal, buccal, transmucosal, transurethral, rectal, nasal, oral, peroral, pulmonary or aural and which is functional, may also be utilized with the present invention.

The solubilizing agent of the present invention comprises a biodegradable ABA-type or BAB-type triblock copolymer having an weight average molecular weight of between 1500 and 3099 consisting of 50.1 to 65% by weight of a hydrophobic A polymer block comprising a biodegradable polyester, and 35 to 49.9% by weight of a hydrophilic B polymer block consisting of polyethylene glycol (PEG), with the proviso that said polymeric composition forms a polymer solution when mixed with an aqueous liquid and remains as a free flowing liquid.

Preferably, the biodegradable polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acids, malic acid, and copolymers thereof. More preferably, the biodegradable polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, and copolymers thereof. Most preferably, the biodegradable polyester is synthesized from monomers selected from the group consisting of D,L-lactide, D-lactide, L-lactide, D,L-lactic acid, D-lactic acid, L-lactic acid, glycolide, glycolic acid, and copolymers thereof.

Polyethylene glycol (PEG) is also sometimes referred to as poly(ethylene oxide) (PEO) or poly(oxyethylene) when incorporated into a triblock copolymer, and the terms can be used interchangeably for the purposes of this invention.

In the hydrophobic A-block, the lactate content is between about 20 to 100 mole percent and is preferably between about 50 to 100 mole percent. The glycolate content is between about 0 to 80 mole percent and is preferably between about 0 to 50 mole percent.

The biodegradable amphiphilic triblock copolymers of the present invention are very effective in solubilizing drugs, particularly hydrophobic drugs, in water to form free flowing solutions. This facilitates administration of a uniform and accurate dose that may then, in many cases, enhance the therapeutic effect of the drug when administered parent rally, particularly intravenously. For the purposes of this invention, the description of the solubilized drug as a solution includes solutions of the drug in the solubilizing media that do not gel at temperatures up to 50° C. Solubilized drugs and drug solutions includes all free flowing forms of the compositions of the present invention. All forms act to facilitate administration of the drug and enhance the therapeutic effect. Such therapeutic effects may be optimized by controlling the copolymer molecular weights, composition, and the relative ratios of the hydrophilic and hydrophobic blocks, ratios of drug to copolymer, and both drug and copolymer concentrations in the final administered dosage form. Additional advantages of this invention will become apparent from the following detailed description of the various embodiments.

### DETAILED DESCRIPTION OF THE INVENTION

This invention is not limited to the particular configurations, process steps, and materials disclosed herein, as such configurations, process steps, and materials may vary somewhat. It is also to be understood that the terminology employed herein is used for the purpose of describing particular embodiments only, and is not intended to be limiting since the scope of the present invention will be limited only by the appended claims and equivalents thereof.

In this specification and the appended claims, the singular forms “a,” “an,” and “the” include plural referents unless the context clearly dictates otherwise. Thus, for example, reference to a composition for delivering “a drug” includes reference to two or more drugs. In describing and claiming the present invention, the following terminology will be used in accordance with the definitions set out below:

“Effective amount” means an amount of a drug or pharmacologically active agent that provides the desired local or systemic effect.

“Polymer solution”, “aqueous solution” and the like, when used in reference to a biodegradable block copolymer contained in such a solution, shall mean a water based solution having such block copolymer contained therein at a functional concentration. Polymer solution includes all free flowing forms of the composition comprising the copolymers of the present invention and water. Polymer solutions act to solubilize the drug in a form that is acceptable for parenteral and particularly for intravenous administration at a physiological relevant temperatures, i.e., 35-42° C.

“Aqueous solution” shall include water without additives, or aqueous solutions containing additives or excipients such as buffer salts, salts for isotonicity adjustment, antioxidants, preservatives, drug stabilizers, etc.

“Drug solution”, “solubilized drug”, and “dissolved drug”, and all other similar terms shall mean a drug in a polymer solution wherein the drug has been solubilized and is free flowing at temperatures relevant for administration, including in many cases administration by the intravenous route. Solubilized drug and drug solution includes all free flowing forms of the compositions comprising the amphiphilic triblock copolymers of the present invention, water and drug(s). The enhancement of dissolution and solubility of the drug leads to advantages in the administration of the drug and attendant enhancement of the therapeutic effect of the drug.

“Parenteral” shall mean administration by means other than through the digestive tract such as by intramuscular, intraperitoneal, intra-abdominal, subcutaneous, intrathecal, intrapleural, intravenous and intraarterial means.

“Intravenous” means administration into a vein.

“Biodegradable” means that the block copolymer can chemically or enzymatically break down or degrade within the body to form nontoxic components. The rate of degradation can be the same or different from the rate of drug release.

“Drug” shall mean any organic or inorganic compound or substance having bioactivity and adapted or used for a therapeutic purpose.

“Hydrophobic drug” shall mean any pharmaceutically beneficial agent having a water solubility of less than 100 mg/mL.

“Peptide,” “polypeptide,” “oligopeptide” and “protein” shall be used interchangeably when referring to peptide or protein drugs and shall not be limited as to any particular molecular weight, peptide sequence or length, field of bioactivity or therapeutic use unless specifically stated.

“PLGA” shall mean a copolymer derived from the condensation copolymerization of lactic acid and glycolic acid, or, by the ring opening polymerization of lactide and glycolide. The terms lactic acid and lactate are used interchangeably; glycolic acid and glycolate are also used interchangeably.

“PLA” shall mean a polymer derived from the condensation of lactic acid or by the ring opening polymerization of lactide.

“Biodegradable polyesters” refer to any biodegradable polyesters, which are preferably synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acids, malic acid, and copolymers thereof.

The present invention is based on the discovery of ABA-type or BAB-type block copolymers, where the A-blocks are relatively hydrophobic polymer blocks comprising biodegradable polyester, and the B-blocks are relatively hydrophilic polymer blocks comprising polyethylene glycol (PEG). The block copolymers have a hydrophobic content of between about 50.1 to 65% by weight and an overall block copolymer weight-averaged molecular weight of between about 1500 and 3099, and which are water soluble and capable of enhancing the solubility of drugs and, fortuitously, hydrophobic drugs, in water, to form a drug solution. It is also within the scope of the invention to include compositions where the drug is solubilized by the copolymer in an aqueous environment, yet the desired dose of the drug exceeds even this enhanced solubility state, and the final formulation of the drug has the visual appearance of a suspension or dispersion, wherein a portion of the total drug load is dissolved and a portion of the total drug load is suspended or dispersed. With such a high hydrophobic content in the block copolymers it is unexpected that such block copolymers would be water soluble. It is also an unexpected discovery that the copolymer of the present invention can significantly increase the water solubility of a hydrophobic drug. Therefore, the biodegradable triblock copolymers of the present invention can be used as solubilizing agents for the delivery of drugs and hydrophobic drugs in particular, and, when administered, the hydrophobic biodegradable polymer blocks decompose by simple hydrolysis in vivo into non-toxic small molecules. A drug may be delivered to a human or any other warm blooded animal much more effectively as an aqueous solution with the biodegradable triblock copolymers of the present invention, thus facilitating administration of a uniform and accurate dose which may then in many cases enhance the therapeutic effect of the drug.

Basic to the present invention is the utilization of a block copolymer having hydrophobic A-block segments and hydrophilic B-block segments. Generally the block copolymer will be an ABA-type or BAB-type triblock copolymer. However, the block copolymer could also be a multiblock copolymer having repeating BA or AB units to make A(BA)n or B(AB)n copolymers where n is an integer from 2 to 5.

Both ABA-type and BAB-type triblock copolymers may be synthesized by ring opening polymerization, or condensation polymerization according to reaction schemes disclosed in U.S. Pat. Nos. 6,004,573 and 6,117,949 and fully incorporated herein by reference.

The subset of block copolymers comprising PEG and PLGA that have utility as disclosed in this invention meet the criteria summarized in Table 1, namely having a compositional make-up within the indicated ranges that result in block copolymers that demonstrate the desired dissolution when exposed to water. For purposes of disclosing molecular weight parameters, all reported molecular weight values are based on measurements by ¹H-NMR or GPC (gel permeation chromatography) analytical techniques. The reported weight average molecular weights and number average molecular weights were determined by GPC and ¹H-NMR, respectively. The reported lactide/glycolide ratio was calculated from ¹H-NMR data. GPC analysis was performed on a Styragel HR-3 column calibrated with PEG standards using RI detection and chloroform as the eluent, or on a combination of Phenogel. ¹H-NMR spectra were taken in CDCl₃ on a Bruker 200 MHz instrument.

TABLE 1

| | |
|---|---|
| Total weight average | 1500 to 3099 |
| molecular weight: | |
| PEG content: | 35 to 49.9% by weight |
| Total polyester content: | 50.1 to 65% by weight |
| Lactate content: | 20 to 100 mole percent |
| Glycolate content: | 0 to 80 mole percent |
| Neat Polymer Behavior: | high viscosity liquid that is water soluble |

The biodegradable, hydrophobic A polymer block(s) comprise a polyester synthesized from monomers selected from the group comprised of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acids, malic acid, and copolymers thereof. The hydrophilic B-block segment is preferably polyethylene glycol (PEG) having a weight average molecular weight of between about 600 and 1500.

Both ABA-type and BAB-type triblock copolymers may be synthesized by ring opening polymerization, or condensation polymerization. For example, the B-blocks may be coupled to the A-blocks by ester or urethane links and the like. Condensation polymerization and ring opening polymerization procedures may be utilized as may the coupling of a monofunctional hydrophilic B block to either end of a difunctional hydrophobic A block in the presence of coupling agents such as isocyanates. Furthermore, coupling reactions may follow activation of functional groups with activating agents, such as carbonyl diimidazole, succinic anhydride, N-hydroxy succinimide and p-nitrophenyl chloroformate and the like.

The hydrophilic B-block is formed from PEG or derivatized PEG of an appropriate molecular weight. PEG was chosen as the hydrophilic, water-soluble block because of its unique biocompatibility, nontoxic properties, hydrophilicity, solubilization properties, and rapid clearance from a patient's body.

The hydrophobic A-blocks are utilized because of their biodegradable, biocompatible, and solubilization properties. The in vitro and in vivo degradation of these hydrophobic, biodegradable polyester A-blocks is well understood and the degradation products are readily metabolized and/or eliminated from the patient's body.

Surprisingly, the total weight percentage of the hydrophobic polyester A-blocks, relative to that of the hydrophilic PEG B-blocks, is high, e.g. between about 50.1 to 65% by weight, yet the resulting triblock copolymer retains its desirable water solubility. It is an unexpected discovery that a block copolymer with such a large proportion of hydrophobic components would be not only water soluble, but also greatly enhance the water solubility of hydrophobic drugs. It is believed that this desirable solubility characteristic is made possible by maintaining an overall weight average molecular weight of the entire triblock copolymer at between about 1500 and 3099. Thus, water soluble biodegradable block copolymers capable of enhancing the water solubility of drugs and especially hydrophobic drugs are prepared wherein the hydrophilic B-block or blocks make up about 35 to 49.9% by weight of the copolymer and the hydrophobic A-block or blocks make up about 50.1 to 65% by weight of the copolymer.

The concentration in an aqueous solution at which the block copolymers are soluble and capable of enhancing the water solubility of a drug, i.e. “polymer solution”, may be considered as the functional concentration. Generally speaking, polymer solutions having block copolymer concentrations of as low as 1% and up to about 50% by weight can be used and still be functional. However, polymer solutions having block copolymer concentrations in the range of about 5 to 40% are preferred and concentrations in the range of about 10 to 30% by weight are most preferred.

Drugs that may be solubilized or dispersed by the block copolymers of the present invention can be any bioactive agent and particularly those having limited solubility or dispersibility in an aqueous or hydrophilic environment, or any bioactive agent that requires enhanced solubility or dispersibility. Without limiting the scope of the present invention, suitable drugs include those drugs presented in the book entitled Goodman and Gilman's The Pharmacological Basis of Therapeutics 9^{th} Edition or the book entitled The Merck Index 12^{th} Edition that both list drugs suitable for numerous types of therapeutic applications, including drugs in the following categories: drugs acting at synaptic and neuroeffector junctional sites, drugs acting on the central nervous system, drugs that influence inflammatory responses, drugs that affect the composition of body fluids, drugs affecting renal function and electrolyte metabolism, cardiovascular drugs, drugs affecting gastrointestinal function, drugs affecting uterine motility, chemotherapeutic agents for parasitic infections, chemotherapeutic agents for microbial diseases, antineoplastic agents, immunosuppressive agents, drugs affecting the blood and blood-forming organs, hormones and hormone antagonists, dermatological agents, heavy metal antagonists, vitamins and nutrients, vaccines, oligonucleotides and gene therapies. Example drugs suitable for use in the present invention include testosterone, testosterone enanthate, testosterone cypionate, methyltestosterone, amphotericin B, nifedipine, griseofulvin, paclitaxel, doxorubicin, daunomycin, indomethacin, ibuprofen, and cyclosporin A.

Incorporating or solubilizing one or more drugs mentioned in the above categories with the block copolymers of the present invention to form an aqueous solution can be achieved by simply adding the drug to an aqueous copolymer mixture, or by mixing the drug with the neat copolymer and thereafter combining the same with water to form a solution.

The mixture of the biodegradable copolymers and peptide/protein drugs, and/or other types of drugs, may be prepared as an aqueous drug delivery liquid. This aqueous drug delivery liquid is then administered parent rally, preferably intravenously. Such formulations may also be suitable for other means of administration such as topically, transdermally, transmucosally, inhaled, or insertion into a cavity such as by ocular, vaginal, transurethral, rectal, nasal, oral, peroral, buccal, pulmonary or aural administration to a patient. In other words, solutions suitable for parenteral, e.g. intravenous, administration may also be administered by any other functional means. However, not all formulation that are suitable for delivery by other means can be delivered intravenously. Alternatively, many aqueous solutions may be further diluted in an i.v. bag or other means, and administered to a patient, without precipitation of the drug for an extended period. This system will cause minimal toxicity and minimal mechanical irritation to the surrounding tissue due to the biocompatibility of the materials, and the A-blocks will be hydrolyzed or biodegraded to corresponding monomers, for example lactic acid, glycolic acid, within a specific time interval.

A distinct advantage of the compositions of this invention lies in the ability of the block copolymer to increase the solubility of many drug substances. The combination of the hydrophobic A-block(s) and hydrophilic B-block(s) renders the block copolymer amphiphilic in nature. This is particularly advantageous in the solubilization of hydrophobic or poorly water-soluble drugs such as cyclosporin A and paclitaxel. What is surprising is the degree of drug solubilization of most, if not all, drugs since the major component of the block copolymer is the hydrophobic A-block content. However, as already discussed, even though hydrophobic polymer block(s) are the major component, the block copolymer is water soluble and it has been found that there is an increase in drug solubility in the presence of the block copolymer.

Another advantage to the composition of the invention lies in the ability of the block copolymer to increase the chemical stability of many drug substances. Various mechanisms for the degradation of drugs, that lead to a drug's chemical instability, have been observed to be inhibited when the drug is in the presence of the block copolymer. For example, paclitaxel and cyclosporin A are substantially stabilized in the aqueous polymer composition of the present invention relative to certain aqueous solutions of these same drugs in the presence of organic co-solvents. This stabilization effect on paclitaxel and cyclosporin A is but illustrative of the effect that can be achieved with many other drug substances.

The biodegradable triblock copolymers of the present invention act as solubilizing agents for drugs and particularly for hydrophobic drugs. In one possible configuration, a dosage form comprised of a solution of the block copolymer that contains dissolved drug is administered to the body. The drug/tri block copolymer solution may be freeze-dried for long-term storage, and the lyophilized biodegradable polymeric drug composition may be restored to its original solution by using water or another predominantly aqueous liquid.

The only limitation as to how much drug can be dissolved into the biodegradable and water soluble triblock copolymer of the present invention is one of functionality, namely, the drug:copolymer ratio may be increased until the drug precipitates, or precipitates when water is added, or the properties of the copolymer are adversely affected to an unacceptable degree, or until the properties of the system are adversely affected to such a degree as to make administration of the system unacceptably difficult. Generally speaking, it is anticipated that in most instances where dissolution is desired, the drug will make up between about 10⁻⁶ to about 100 percent by weight of the copolymer with ranges between about 0.001% to 25% by weight being most common. For example, the drug being present at 100% by weight of the copolymer means the drug and copolymer are present in equal amounts (i.e., equal weights). Generally speaking, it is anticipated that in most instances where a drug dispersion is desired, the upper range of drug:copolymer ratios could substantially exceed the range noted above for dissolution. These ranges of drug loading are illustrative and will include most drugs that may be utilized in the present invention. However, such ranges are not limiting to the invention should drug loadings outside this range be functional and effective.

The present invention thus provides a biodegradable polymeric solubilizing agent for drugs and preferably hydrophobic drugs. The drug solution formed with the biodegradable polymeric solubilizing agent of the present invention has demonstrates the desired physical stability, therapeutic efficacy, and toxicology.

In order to illustrate preferred embodiments of this invention, the synthesis of various low molecular weight ABA-type or BAB-type block copolymers consisting of 50.1 to 65% by weight hydrophobic A-blocks (biodegradable polyesters), and 35 to 49.9% by weight hydrophilic B-blocks (polyethylene glycol “PEG”) were completed. The objective was to prepare of ABA or BAB triblock copolymers having weight average molecular weights of about 1500 to 3099. In the case where each A-block consists of a biodegradable polyester synthesized from monomers selected from the group consisting of D,L-lactide, D-lactide, L-lactide, D,L-lactic acid, D-lactic acid, L-lactic acid, glycolide, or glycolic acid, the composition of the A-block is about 20 to 100 mole percent lactate and 0 to 80 mole percent glycolate.

The following are examples that illustrate preferred embodiments of the invention but are intended as being representative only.

### EXAMPLES

### Example 1

Synthesis of the ABA-type Triblock Copolymer PLGA-PEG-PLGA by Ring Opening Copolymerization

PEG (Mw=1000; 100 g ) was dried under vacuum (1 mmHg) at 100° C. for 5 hours. D,L-Lactide (86.72 grams)and glycolide (23.28 grams) were added to the flask and heated to 130° C. to afford a homogenous solution. Polymerization was initiated by the addition of 40 mg stannous octoate to the reaction mixture. After maintaining the reaction for five hours at 155° C., the reaction was stopped and the flask was cooled to room temperature. Unreacted lactide and glycolide were removed by vacuum distillation for 2 hours at 130° C. The raw copolymer residue was a high viscosity liquid. The copolymer was purified twice by dissolving it in water to afford a 25% solution, and letting the solution stir overnight at room temperature followed by raising the solution temperature to 70° C. to precipitate the polymer. The supernatant was decanted from the flask. Any water remaining was removed by freeze drying. The resulting PLGA-PEG-PLGA copolymer had a weight averaged molecular weight (Mw) of 2324 as measured by GPC. The GPC was performed on two Phenogel columns (300×7.8), at 500 Å, and with a mixed bed connected in series. The mobile phase was tetrahydrofuran. Calibration was with PEG standards. Detection was by refractive index. In addition, the resulting copolymer formed a polymer solution when mixed with an aqueous liquid and remained as a free flowing liquid at temperatures up to 50° C.

### Example 2

Following the basic procedure outlined in Example 1, other triblock copolymers were synthesized using PEG (Mw=600, 1000, or 1450) with various lactide and/or glycolide contents. The properties of these triblock copolymers were listed in the following table:

EXAMPLE ABA
Block Copolymers with
Solubilizing Enhancing Function

| | | PLGA/PEG or | | Solubilizing |
|---|---|---|---|---|
| | PEG Molecular | PLA/PEG weight | LA:GA | Enhancing |
| Entry | Weight | Ratio | (mole ratio) | Function |
| 1 | 600 | 1.1 | 75:25 | Yes |
| 2 | 1000 | 1.1 | 75:25 | Yes |
| 3 | 1450 | 1.1 | 75:25 | Yes |
| 4 | 600 | 1.1 | 100:0 | Yes |
| 5 | 1000 | 1.1 | 100:0 | Yes |
| 6 | 1450 | 1.1 | 100:0 | Yes |

It was noted that all of the block copolymers listed in the above table possessed the property of enhancing the solubility of drugs and particularly of hydrophobic drugs. Hence, both PLGA-PEG-PLGA and PLA-PEG-PLA triblock copolymers were prepared and the results are summarized in this example. The copolymers formed polymer solutions when mixed with an aqueous liquid and remained as free flowing liquids.

### Example 3

The solubility enhancing properties of aqueous solutions of the ABA triblock copolymer of Example 1 are illustrated in this example. Polymer solutions containing 23% by weight of the copolymer were prepared in water, and paclitaxel was added to the solution and the mixture was stirred for approximately 20 minutes. The mixture was then filtered through a 0.2 μm filter to give a clear solution that was analyzed for paclitaxel content and hence aqueous solubility. The aqueous solubility of paclitaxel was enhanced from approximately 5 μg/ml in pure water to greater than 25 mg/ml in the 23% by weight aqueous solution of the triblock copolymer. The solubility of paclitaxel was increased by at least 5000-fold. The ABA triblock copolymeric composition formed a polymer solution containing paclitaxel when mixed with an aqueous liquid and remained as a free flowing liquid.

### Example 4

Cyclosporin A is another hydrophobic drug that is highly insoluble in water (solubility is approximately 4 μg/ml in pure water). Thus, cyclosporin A (4 mg) was mixed with 600 mg of polymer prepared by the method described in Example 1, along with 2 ml water to afford a clear solution without any undissolved particles present. There was at least a 400-fold increase in the solubility of cyclosporin A. The ABA triblock copolymeric composition formed a polymer solution containing cyclosporin A when mixed with an aqueous liquid and remained as a free flowing liquid.

### Example 5

This example illustrates the solubility enhancing effect of the triblock copolymers of the present invention on the hydrophobic drugs nifedipine and griseofulvin. The water solubilities of nifedipine and griseofulvin were 6 μg/mL and 10 μg/mL, respectively.

Triblock copolymers of Example 2 were used. The neat polymer and the drug were mixed and gently heated (ca. 50° C.) to completely dissolve the drug. Water was added to the mixture to afford a 23% by weight aqueous solution of the triblock copolymers. The solution was allowed to stand for 30 minutes before filtration (0.2 μm pore size filter). The solubilities of nifedipine and griseofulvin in various triblock copolymer solutions of the present invention were measured as shown in the following table:

| | | | Drug Solubility | |
|---|---|---|---|---|
| | | | (mg/ml in 23% | |
| Copolymer | | | w/w copolymer | |
| | PLGA/PEG | | solution, 25° C.) | |
| PEG MW | Wt. Fraction | L/G Mole fraction | Nifedipine | Griseofulvin |
| 1450 | 1.1 | 75/25 | 3.74 | 1.50 |
| 1000 | 1.1 | 75/25 | 3.70 | 1.5 |

The results show that various triblock copolymers of the present invention increased the solubilities of griseofulvin and nifedipine by approximately 100 and 1000 fold, respectively. The triblock copolymeric compositions formed polymer solutions containing nifedipine or griseofulvin when mixed with an aqueous liquid and remained as free flowing liquids.

### Example 6

This example illustrates the solubility enhancing effect of the triblock copolymers of the present invention on the hydrophobic drug amphotericin B.

The triblock copolymer of Example 2 (entry number 2) was used. The drug was mixed with the copolymer solution (23 wt % copolymer in water). The mixture was allowed to stand for 30 minutes before filtration. The reported solubility of amphotericin B in pure water is 3 μg/mL. The solubility of amphotericin B in the aqueous triblock copolymer solution of the present invention was 150 μg/mL. The present invention increased the solubility of amphotericin B by 50-fold. The copolymeric composition formed a polymer solution containing amphotericin B when mixed with an aqueous liquid and remained as a free flowing liquid.

### Example 7

BAB-type triblock copolymers were synthesized by coupling two methoxy-PEG-PLGA diblocks using hexyl diisocynate where the PEG B-block at either end has a Mw of 750 and the A-block has a combined molecular weight of about 1500 with various lactide and/or glycolide contents. Although diblocks can be coupled via ester or urethane linkages, or a combination of ester and urethane linkages, the copolymers of this example contained urethane linkages. The properties of these triblock copolymers are listed in the following table.

EXAMPLE BAB
Triblock Copolymers with Solubility
Enhancing Function

| | | | Solubilizing |
|---|---|---|---|
| Weight-Averaged | Weight % | PLA:PGA | Enhancing |
| Molecular Weight | A-blocks | (mole ratio) | Function |
| 2640 | 50.1 | 50:50 | Yes |
| 2640 | 50.1 | 100:0 | Yes |

All of the PEG-PLGA-PEG triblock copolymers, namely BAB-type triblock copolymers listed in the above table show the solubility enhancing function. The copolymeric composition forms a polymer solution containing drug when mixed with an aqueous liquid and remains as a free flowing liquid.

### Example 8

This example illustrates the aqueous stability enhancing effect of the triblock copolymers of the present invention on the hydrophobic drug paclitaxel. The triblock copolymer of Example 2 (entry number 2) was used. Paclitaxel was dissolved into acetonitrile, acetonitrile:water (50:50, v/v), or triblock copolymer and incubated at 40° C. for 7 days. The paclitaxel concentration at day 7 decreased by 8.5, 4, and 90% for solutions of triblock copolymer, acetonitrile, and acetonitrile:water (50:50), respectively, in comparison to day 0. The triblock copolymers of the present invention increased the stability of paclitaxel in an aqueous system by 10-fold.

EXAMPLE ABA
Triblock Copolymers with Solution
Stability Enhancing Function

| | | Day 1 | Day 3 | Day 7 |
|---|---|---|---|---|
| Solution | Day 0 (mg/mL) | (mg/mL) | (mg/mL) | (mg/mL) |
| Example2 | 13.1 | 13.5 | 13.4 | 12.0 |
| (entry number 2) | | | | |
| Acetonitrile | 9.2 | 9.2 | 9.1 | 8.9 |
| Acetonitrile:water | 0.2 | 0.15 | 0.08 | 0.02 |
| (50:50) | | | | |

### Example 9

This example illustrates the enhancing effect of the triblock copolymers of the present invention to prevent precipitation of the solubilized hydrophobic drugs paclitaxel and cyclosporin A from examples 3 and 4, respectively, upon dilution. The triblock copolymer of Example 2 (entry number 2) was used. Following preparation of the paclitaxel and cyclosporin A solutions from examples 3 and 4, a portion of each solution was diluted 10, 100, and 1000-fold with water. The drugs remained in solution for all diluted solutions, i.e., no evidence of precipitation, for greater than 24 hours.

The above description will enable one skilled in the art to make ABA-type (e.g., PLGA-PEG-PLGA and PLA-PEG-PLA) or BAB-type (e.g., PEG-PLGA-PEG and PEG-PLA-PEG) triblock copolymers that enhance the solubility of hydrophobic drugs and can be used as biodegradable and biocompatible solubilizing agents in the field of drug delivery. Although the enhanced solubility of a few hydrophobic drugs are illustrated in the examples to show the functionality of the triblock copolymers of the present invention, these descriptions are not intended to be an exhaustive statement of all drugs whose solubility can be enhanced by the biodegradable block copolymers of the present invention. Certainly, numerous other drugs from various categories of therapeutic agents are well suited for forming aqueous solutions with the triblock copolymers as described in this invention. Neither are all block copolymers shown which may be prepared, and which demonstrate the property of enhancing the solubility of a drug. However, it will be immediately apparent to one skilled in the art that various modifications may be made without departing from the scope of the invention.

We claim:

1. A polymeric composition having improved capability to solubilize a drug in a hydrophilic environment, comprising: a biodegradable ABA-type, or BAB-type block copolymer, comprising:
i) 50.1 to 65% by weight of a biodegradable, hydrophobic A polymer block comprising a biodegradable polyester, and
ii) 35 to 49.9% by weight of a hydrophilic B polymer block comprising a polyethylene glycol (PEG), and wherein the block copolymer has a weight averaged molecular weight of between 1500 to 3099 Daltons,
with the proviso that said polymeric composition when formed as an aqueous polymer solution, is a free flowing liquid at body temperatures.

2. The polymeric composition according to claim 1 wherein the biodegradable polyester of the hydrophobic A polymer block is synthesized from monomers selected from the group consisting of D, L-lactide, D-lactide, L-lactide, D, L-lactic acid, D-lactic acid, L-lactic acid, glycolide, glycolic acid, ε-caprolactone, ε-hydroxy hexanoic acid, and copolymers thereof.

3. The polymeric composition according to claim 1 wherein the A polymer block comprises between about 20 to 100 mole percent lactide or lactic acid, and between about 0 to 80 mole percent glycolide or glycolic acid.

4. A biodegradable polymeric drug delivery composition capable of solubilizing a drug in a hydrophilic environment to form a solution, comprising:
(a) an effective amount of a drug; and
(b) a biodegradable ABA-type, or BAB-type block copolymer comprising:
i) 50.1 to 65% by weight of a biodegradable, hydrophobic A polymer block comprising a biodegradable polyester, and
ii) 35 to 49.9% by weight of a hydrophilic B polymer block comprising a polyethylene glycol (PEG), and wherein the block copolymer has a weight-averaged molecular weight of between 1500 to 3099 Daltons,
wherein said composition forms a free flowing liquid at body temperatures in an aqueous environment.

5. The polymeric drug delivery composition according to claim 4 wherein the biodegradable polyester of the hydrophobic A polymer block is synthesized from monomers selected from the group consisting of D, L-lactide, D-lactide, L-lactide, D, L-lactic acid, D-lactic acid, L-lactic acid, glycolide, glycolic acid, ε-caprolactone, ε-hydroxy hexanoic acid, and copolymers thereof.

6. The polymeric drug delivery composition according to claim 4 wherein the A polymer block comprises between about 20 to 100 mole percent lactide or lactic acid, and between about 0 to 80 mole percent glycolide or glycolic acid.

7. The polymeric drug delivery composition according to claim 4 wherein the drug content is 10⁻⁶ to 100% of the total triblock copolymer weight.

8. A biodegradable polymer solution as a drug delivery vehicle capable of solubilizing a drug in a hydrophilic environment, comprising: a functional concentration of a biodegradable ABA-type, or BAB-type block copolymer and an aqueous solution, said block copolymer comprising:
i) 50.1 to 65% by weight of a biodegradable, hydrophobic A polymer block comprising a biodegradable polyester, and
ii) 35 to 49.9% by weight of a hydrophilic B polymer block comprising a polyethylene glycol (PEG), and wherein the block copolymer has a weight-averaged molecular weight of between 1500 to 3099 Daltons;
and wherein said polymer solution is a free flowing liquid at body temperatures.

9. The polymeric solution according to claim 8 wherein said functional concentration of said copolymer is between about 1 to 50% by weight of said polymer solution.

10. The polymeric composition according to claim 8 wherein the biodegradable polyester of the hydrophobic A polymer block is synthesized from monomers selected from the group consisting of D, L-lactide, D-lactide, L-lactide, D, L-lactic acid, D-lactic acid, L-lactic acid, glycolide, glycolic acid, ε-caprolactone, ε-hydroxy hexanoic acid, and copolymers thereof.

11. The polymeric composition according to claim 8 wherein the A polymer block comprises between about 20 to 100 mole percent lactide or lactic acid, and between about 0 to 80 mole percent glycolide or glycolic acid.

12. A biodegradable drug solution comprising:
(a) an effective amount of a drug solubilized in a polymer solution comprising;
(1) a functional concentration of a biodegradable ABA-type, or BAB-type block copolymer capable of solubilizing said drug in a hydrophilic environment, comprising:
i) 50.1 to 65% by weight of a biodegradable, hydrophobic A polymer block comprising a biodegradable polyester, and
ii) 35 to 49.9% by weight of a hydrophilic B polymer block comprising a polyethylene glycol (PEG), and wherein the tri-block copolymer has a weight-averaged molecular weight of between 1500 to 3099 Daltons; and
(2) an aqueous solution,
with the proviso that said polymer solution is a free flowing liquid at a body temperature.

13. The biodegradable aqueous polymeric drug solution according to claim 12 further comprising excipients, additives, buffers, osmotic pressure adjusting agents, antioxidants, preservatives, drug stabilizing agents or equivalents thereof.

14. The biodegradable aqueous polymeric drug solution according to claim 12 wherein the functional concentration of said copolymer is between about 1 to 50% by weight of said polymer solution.

15. The biodegradable aqueous polymeric drug solution according to claim 12 wherein the drug content is 10⁻⁶ to 100% of the total triblock copolymer weight.

16. The biodegradable aqueous polymeric drug solution according to claim 12 wherein the biodegradable polyester of the hydrophobic A polymer block is synthesized from monomers selected from the group consisting of D, L-lactide, D-lactide, L-lactide, D, L-lactic acid, D-lactic acid, L-lactic acid, glycolide, glycolic acid, ε-caprolactone, ε-hydroxy hexanoic acid, and copolymers thereof.

17. The biodegradable aqueous polymeric drug solution according to claim 12 wherein the A-block comprises between about 20 to 100 mole percent lactide or lactic acid and between about 0 to 80 mole percent glycolide or glycolic acid.

18. A method for administering a drug to a warm blooded animal, comprising
(1) providing a biodegradable polymeric drug delivery composition comprising:
(a) an effective amount of a drug; and
(b) a biodegradable ABA-type, or BAB-type block copolymer comprising:
i) 50.1 to 65% by weight of a biodegradable, hydrophobic A polymer block comprising a biodegradable polyester, and
ii) 35 to 49.9% by weight of a hydrophilic B polymer block comprising a polyethylene glycol (PEG), and wherein the block copolymer has a weight-averaged molecular weight of between 1500 to 3099 Daltons,
with the proviso that said polymeric composition forms a free flowing liquid at body temperature in an aqueous environment, and
(2) administering said composition to said warm blooded animal.

19. The method according to claim 18 wherein the biodegradable polyester of the hydrophobic A polymer block is synthesized from monomers selected from the group consisting of D, L-lactide, D-lactide, L-lactide, D, L-lactic acid, D-lactic acid, L-lactic acid, glycolide, glycolic acid, ε-caprolactone, ε-hydroxy hexanoic acid, and copolymers thereof.

20. The method according to claim 18 wherein the A polymer block comprises between about 20 to 100 mole percent lactide or lactic acid, and between about 0 to 80 mole percent glycolide or glycolic acid.

21. The method according to claim 18 wherein the drug content is 10⁻⁶ to 100% of the total triblock copolymer weight.

22. The method according to claim 18 wherein said administration is by parenteral, ocular, topical, inhalation, transdermal, vaginal, buccal, transmucosal, transurethral, rectal, nasal, oral, peroral, pulmonary or aural means.

23. A method for administering a drug to a warm blooded animal, comprising
(1) providing a biodegradable polymeric drug solution comprising an effective amount of a drug solubilized in a polymer solution comprising;
(a) a functional concentration of a biodegradable ABA-type, or BAB-type block copolymer capable of solubilizing said drug in a hydrophilic environment, comprising:
i) 50.1 to 65% by weight of a biodegradable, hydrophobic A polymer block comprising a biodegradable polyester, and
ii) 35 to 49.9% by weight of a hydrophilic B polymer block comprising a polyethylene glycol(PEG), and wherein the tri-block copolymer has a weight-averaged molecular weight of between 1500 to 3099 Daltons; and
(b) an aqueous solution; with the proviso that said polymer solution is a free flowing liquid at body temperatures, and;
(2) administering said drug solution to said warm blooded animal.

24. The method according to claim 23 wherein the functional concentration of said copolymer is between about 1 to 50% by weight of said polymer solution.

25. The method according to claim 23 wherein the biodegradable polyester of the hydrophobic A polymer block is synthesized from monomers selected from the group consisting of D, L-lactide, D-lactide, L-lactide, D, L-lactic acid, D-lactic acid, L-lactic acid, glycolide, glycolic acid, ε-caprolactone, ε-hydroxy hexanoic acid, and copolymers thereof.

26. The method according to claim 23 wherein the A-block comprises between about 20 to 100 mole percent lactide or lactic acid and between about 0 to 80 mole percent glycolide or glycolic acid.

27. The method according to claim 23 wherein the drug content is 10⁻⁶ to 100% of the total triblock copolymer weight.

28. The method according to claim 23 wherein said administration is by intramuscular, intraperitoneal, intra-abdominal, subcutaneous, intrathecal, intrapleural, intravenous or intraarterial means.

29. A method for enhancing the solubility of a drug, comprising
1) preparing a polymeric composition comprising a functional concentration of a biodegradable ABA-type, or BAB-type block copolymer, comprising:
i) 50.1 to 65% by weight of a biodegradable, hydrophobic A polymer block comprising a biodegradable polyester, and
ii) 35 to 49.9% by weight of a hydrophilic B polymer block comprising a polyethylene glycol (PEG), and wherein the block copolymer has a weight averaged molecular weight of between 1500 to 3099 Daltons,
2) admixing the polymeric composition with a drug; and
3) admixing the drug containing polymeric composition with an aqueous solution to obtain a drug solution that remains a free flowing liquid at body temperatures.

30. The method according to claim 29 wherein the functional concentration of said copolymer is between about 1 to 50% by weight of said polymer solution.

31. The method according to claim 29 wherein the biodegradable polyester of the hydrophobic A polymer block is synthesized from monomers selected from the group consisting of D, L-lactide, D-lactide, L-lactide, D, L-lactic acid, D-lactic acid, L-lactic acid, glycolide, glycolic acid, ε-caprolactone, ε-hydroxy hexanoic acid, and copolymers thereof.

32. The method according to claim 31 wherein the A-block comprises between about 20 to 100 mole percent lactide or lactic acid and between about 0 to 80 mole percent glycolide or glycolic acid.

33. The method according to claim 29 wherein the drug content is 10⁻⁶ to 100% of the total triblock copolymer weight.

34. A method for enhancing the solubility of a drug, comprising
1) preparing a polymeric composition comprising a functional concentration of a biodegradable ABA-type, or BAB-type block copolymer, comprising:
i) 50.1 to 65% by weight of a biodegradable, hydrophobic A polymer block comprising a biodegradable polyester, and
ii) 35 to 49.9% by weight of a hydrophilic B polymer block comprising a polyethylene glycol (PEG), and wherein the block copolymer has a weight averaged molecular weight of between 1500 to 3099 Daltons,
2) admixing said composition with an aqueous solution to form a polymeric solution that remains a free flowing liquid at body temperatures, and
3) admixing said polymer solution with a drug to form a drug solution.

35. The method according to claim 34 wherein the functional concentration of said copolymer is between about 1 to 50% by weight of said polymer solution.

36. The method according to claim 34 wherein the biodegradable polyester of the hydrophobic A polymer block is synthesized from monomers selected from the group consisting of D, L-lactide, D-lactide, L-lactide, D, L-lactic acid, D-lactic acid, L-lactic acid, glycolide, glycolic acid, ε-caprolactone, ε-hydroxy hexanoic acid, and copolymers thereof.

37. The method according to claim 34 wherein the A-block comprises between about 20 to 100 mole percent lactide or lactic acid and between about 0 to 80 mole percent glycolide or glycolic acid.

38. The method according to claim 34 wherein the drug content is 10⁻⁶ to 100% of the total triblock copolymer weight.

39. A method for enhancing the solubility of a drug, comprising
1) preparing a polymeric composition comprising a functional concentration of a biodegradable ABA-type, or BAB-type block copolymer, comprising:
i) 50.1 to 65% by weight of a biodegradable, hydrophobic A polymer block comprising a biodegradable polyester, and
ii) 35 to 49.9% by weight of a hydrophilic B polymer block comprising a polyethylene glycol (PEG), and wherein the block copolymer has a weight averaged molecular weight of between 1500 to 3099 Daltons,
2) admixing a drug with an aqueous solution to form a drug-aqueous solution mixture, and
3) admixing said polymer composition with said drug-aqueous solution mixture to form a drug polymeric solution that remains as a free flowing liquid at a body temperature.

40. The method according to claim 39 wherein the functional concentration of said copolymer is between about 1 to 50% by weight of said polymer solution.

41. The method according to claim 39 wherein the biodegradable polyester of the hydrophobic A polymer block is synthesized from monomers selected from the group consisting of D, L-lactide, D-lactide, L-lactide, D, L-lactic acid, D-lactic acid, L-lactic acid, glycolide, glycolic acid, ε-caprolactone, ε-hydroxy hexanoic acid, and copolymers thereof.

42. The method according to claim 39 wherein the A-block comprises of between about 20 to 100 mole percent lactide or lactic acid and between about 0 to 80 mole percent glycolide or glycolic acid.

43. The method according to claim 39 wherein the drug content is 10⁻⁶ to 100% of the total tri block copolymer weight.
